(19) United States
(12) Patent Application Publication
(10) Pub. No.: US 2025/0074859 A1
(43) Pub. Date: Mar. 6, 2025

(54) PROCESS FOR THE CONTINUOUS DISTILLATION OF ACRYLATES

(71) Applicant: BASF SE, Ludwigshafen am Rhein (DE)

(72) Inventors: Tile GIESHOFF, Ludwigshafen am Rhein (DE); Marvin KRAMP, Ludwigshafen am Rhein (DE); Cornelis Hendricus DE RUITER, Ludwigshafen am Rhein (DE); Karl-Friedrich SCHNEIDER, Ludwigshafen am Rhein (DE); Felix HUELSMANN, Ludwigshafen am Rhein (DE); Christian REIN, Ludwigshafen am Rhein (DE); David ELIXMANN, Ludwigshafen am Rhein (DE)

(21) Appl. No.: 18/292,365

(22) PCT Filed: Jul. 19, 2022
(86) PCT No.: PCT/EP2022/070157

§ 371 Date: Jan. 26, 2024

(30) Foreign Application Priority Data
Jul. 29, 2021 (EP) 21188534.8

(51) Int. Cl. C07C 67/54

(52) U.S. Cl. CPC C07C 67/54

(57) ABSTRACT

A process for continuously distiling acrylates by means of a rectification column, wherein the acrylate content in the feed to the rectification column is at least 80% by weight, the liquid in the bottom region of the rectification column is heated by means of an evaporator, and the parts of the evaporator that are in contact with product are made from stainless steel.

The present invention relates to a process for continuously distilling acrylates by means of a rectification column, wherein the acrylate content in the feed to the rectification column is at least 80% by weight, the liquid in the bottom region of the rectification column is heated by means of an evaporator, and the parts of the evaporator that are in contact with product are made from stainless steel.

The polymers and copolymers prepared on the basis of acrylates are of great economic significance in the form of polymer dispersions. They find use, for example, as adhesives, paints, or textile, leather and papermaking assistants.

JP H01-180850 A describes the influence of surface roughness on the formation of polymer in distillation columns.

JP 2001-213844 A describes the preparation of acrylates and methacrylates. For avoidance of corrosion, for example as a result of the acidic catalysts used, alloys composed of 6% to 20% by weight of nickel, 14% to 24% by weight of chromium and 0.5% to 5.5% by weight of cobalt are proposed.

WO 2005/040084 A1 describes the use of alloys having sufficient copper for avoidance of polymerization of ethylenically unsaturated monomers.

Acrylates are typically prepared by esterification of acrylic acid. The acrylates obtained in the reaction are subsequently distilled. For the purifying distillation, rectification columns having evaporators made from nonalloyed steels are used here.

Solid-state deposits (fouling) are formed on the evaporator surfaces, which can hinder heat transfer and even lead to blockages. These solid-state deposits regularly have to be removed mechanically.

The object was accordingly that of finding an improved process for distillation of acrylates, especially with a lower level of solid-state deposits in the evaporators used.

The object is achieved by a process for continuously distilling acrylates by means of a rectification column, wherein the acrylate content in the feed to the rectification column is at least 80% by weight, the liquid in the bottom region of the rectification column is heated by means of an evaporator, and the parts of the evaporator that are in contact with product are made from stainless steel.

Suitable acrylates are, for example, methyl acrylate, ethyl acrylate, n-butyl acrylate and 2-ethylhexyl acrylate. Acrylates are also referred to as acrylic esters.

The acrylate content in the feed to the rectification column is preferably at least 85% by weight, even more preferably at least 90% by weight, even more preferably at least 95% by weight.

The feed to the rectification column preferably has an acid number of less than 100 mg of potassium hydroxide per g of feed, more preferably of less than 10 mg of potassium hydroxide per g of feed, most preferably of less than 1 mg of potassium hydroxide per g of feed. For determination of the acid number, 1 g of feed is diluted with 100 ml of ethanol and titrated with 0.1 molar potassium hydroxide in ethanol using phenolphthalein.

The rectification column is of a design known per se and consists of the actual column body with the separating internals, an evaporator in the bottom region of the rectification column, and a condenser in the top region of the rectification column. In the continuous distillation, feed is continuously metered into the rectification column, and distilled acrylate is continuously drawn off.

The separating internals used may in principle be all standard internals, for example trays, structured packings and/or random packings. Of the trays, preference is given to bubble-cap trays, sieve trays, valve trays, Thormann trays and/or dual-flow trays; of the random packings, preference is given to those comprising rings, helices, saddles, Raschig, Intos or Pall rings, barrel or Intalox saddles, Top-Pak etc., or braids.

The evaporator is of a design known per se. It is in the parts of the evaporator that are in contact with product that the transfer of heat from the evaporator to the liquid to be evaporated takes place. Suitable evaporators are, for example, shell and tube heat exchangers. A shell and tube heat exchanger consists of a shell space and a tube space. The heating medium flows through the shell space. In the case of evaporators, the heating medium is typically heating steam, which condenses on the outside of tubes in the shell space. The liquid to be evaporated flows through the tube space, which consists of many tubes. The insides of the tubes here are the parts of the evaporator that are in contact with product.

The shell and tube heat exchanger may be operated as an internal or external evaporator. An internal evaporator is present directly beneath the separating internals in the rectification column. An external evaporator is present alongside the rectification column and is connected to the lower region of the rectification column. The circulation through an external evaporator can be boosted by a pump (forced circulation evaporator). It is possible to incorporate a pressureretaining valve into the reflux of a forced circulation evaporator. This avoids boiling in the shell and tube heat exchanger, and evaporation takes place only on expansion into the lower region of the rectification column (forced circulation flash evaporator). The latter is particularly gentle.

The condenser is likewise of a design known per se. The condenser may be operated as an internal or external condenser. An external condenser is present alongside the rectification column and is connected to the upper region of the rectification column. Suitable condensers are, for example, shell and tube heat exchangers. A shell and tube heat exchanger consists of a shell space and a tube space. The cooling medium flows through the shell space. The gas to be condensed flows through the tube space, which consists of many tubes. A portion of the condensed gas is recycled as reflux into the rectification column.

The parts of the evaporator that are in contact with product are made from stainless steel. Stainless steels in the context of this invention are steels having iron as the main constituent and at least 10.5% by weight of chromium.

The preferred stainless steels preferably contain 10.5% to 30.0% by weight, more preferably 16.0% to 26% by weight, especially preferably 17.0% to 20.5% by weight, most preferably 18.0% to 20.0% by weight, of chromium, and more preferably additionally preferably 2.0% to 35.0% by weight, more preferably 8.0% to 26.0% by weight, especially preferably 10.0% to 25.0% by weight, most preferably 12.0% to 24.0% by weight, of nickel, and/or additionally preferably 0.1% to 8.0% by weight, more preferably 2.0% to 5.0% by weight, especially preferably 2.5% to 4.5% by weight, most preferably 3.0% to 4.0% by weight, of molybdenum.

The present invention is based on the finding that the use of stainless steels can distinctly reduce the formation of solid-state deposits.

Streams of matter having a high acrylate content that are obtained in the preparation of acrylates are not corrosive. In the purifying distillation of acrylates, therefore, rectification columns and evaporators made of nonalloyed steel are used. No significant loss of material through corrosion is to be expected. It is possibly the case that very small traces of iron are nevertheless dissolved, which then promote the formation of solid-state deposits.

The preparation of the acrylates is described hereinafter:

Acrylates are prepared in various ways in a manner known per se through esterification of acrylic acid with an alcohol, e.g. an alkanol. Acrylates are generally obtained via a homogeneously or heterogeneously catalyzed esterification, as described, for example, in Kirk Othmer, Encyclopedia of Chemical Technology, 4th ed., 1994, pages 301-302 and Ullmann's Encyclopedia of Industrial Chemistry, 5th edition, volume A1, pages 167-169.

The literature includes numerous processes for preparing acrylates by esterification of acrylic acid with an alcohol, for example in DE 196 04 252 A1 and DE 196 04 253 A1. A process for preparing n-butyl acrylate by acid-catalyzed esterification of acrylic acid with n-butanol is disclosed, for example, in WO 98/52904. One example of a batchwise acid-catalyzed esterification is EP 0 890 568 A1.

The alcohol used is typically any alcohol comprising 1 to 12 carbon atoms, for example monoor polyhydric alcohols, preferably mono to tetrahydric, more preferably mono to trihydric, most preferably mono- or dihydric and especially monohydric.

Examples are methanol, ethanol, n-propanol, isopropanol, n-butanol, sec-butanol, isobutanol, tert-butanol, ethylene glycol monomethyl ether, ethylene glycol monoethyl ether, diethylene glycol, propane-1,3-diol monomethyl ether, propane-1,2-diol, ethylene glycol, 2,2-dimethylethane1,2-diol, propane-1,3-diol, butane-1,2-diol, butane-1,4-diol, dimethylaminoethanol, n-hexanol, nheptanol, n-octanol, n-decanol, n-dodecanol, 2-ethylhexanol, 3-methylpentane-1,5-diol, 2-ethylhexane-1,3-diol, 2,4-diethyloctane-1,3-diol, hexane-1,6-diol, cyclopentanol, cyclohexanol, cyclooctanol, cyclododecanol, triethylene glycol, tetraethylene glycol, pentaethylene glycol, npentanol, stearyl alcohol, cetyl alcohol, lauryl alcohol, trimethylolbutane, trimethylolpropane, trimethylolethane, neopentyl glycol and the ethoxylated and propoxylated conversion products thereof, neopentyl glycol hydroxypivalate, pentaerythritol, 2-ethylpropane-1,3-diol, 2-methylpropane-1,3-diol, 2-ethylhexane-1,3-diol, glycerol, ditrimethylolpropane, dipentaerythritol, hydroquinone, bisphenol A, bisphenol F, bisphenol B, bisphenol S, 5-methyl-5-hydroxymethyl-1,3-dioxane, 2,2-bis(4-hydroxycyclohexyl) propane, cyclohexane-1,1-, -1,2-, -1,3-and-1,4-dimethanol, cyclohexane-1,2-, -1,3- or 1,4-diol.

Preferred alcohols are methanol, ethanol, n-butanol, isobutanol, sec-butanol, 2-ethylhexyl alcohol, n-octanol and dimethylaminoethanol. Particularly preferred alcohols are methanol, ethanol, n-butanol, 2-ethylhexyl alcohol and dimethylamino alcohol.

Very particularly preferred alcohols are methanol, ethanol, n-butanol, and 2-ethylhexyl alcohol.

The usable acidic catalysts are preferably sulfuric acid, p-toluenesulfonic acid, benzenesulfonic acid, dodecylbenzenesulfonic acid, methanesulfonic acid or mixtures thereof; also conceivable are acidic ion exchangers or zeolites.

Particular preference is given to using sulfuric acid, p-toluenesulfonic acid and methanesulfonic acid; very particular preference is given to sulfuric acid and p-toluenesulfonic acid.

The catalyst concentration based on the reaction mixture is, for example, 1% to 20% by weight, preferably 5% to 15% by weight.

The preparation of acrylates by transesterification in the presence of acidic or basic catalysts is common knowledge (Ullmann's Encyclopedia of Industrial Chemistry, 5th edition, volume A1, page 171).

There are numerous examples in the literature of transesterifications for preparation of acrylates from acrylates with alcohols, for example the preparation of dimethylaminoethyl acrylate by transesterification of methyl acrylate with dimethylaminoethanol in EP 0 906 902 A2. A batchwise transesterification is described, for example, in EP 1 078 913 A2.

Catalysts proposed are in particular titanium alkoxides wherein the alkyl groups are C₁-C₄-alkyl radicals, e.g. tetramethyl, tetraethyl, tetraisopropyl, tetrapropyl, tetraisobutyl and tetrabutyl titanate (see EP 1 298 867 B1, EP 0 960 877 A2). Further titanium compounds are also described in DE 101 27 939 A1. Also among the catalysts proposed are titanium phenoxides (DE 200 86 18 A1), dibutyltin oxide (EP 0 906 902 A2), metal chelate compounds of, for example, hafnium, titanium, zirconium or calcium, alkali metal and magnesium alkoxides, organic tin compounds or calcium and lithium compounds, for example oxides, hydroxides, carbonates or halides.

Suitable polymerization inhibitors may, for example, be N-oxides (nitroxyl or N-oxyl free radicals, i.e. compounds having at least one >N—O— group), for example 4-hydroxy-2,2,6,6-tetramethylpiperidine N-oxyl or 4-oxo-2,2,6,6-tetramethylpiperidine N-oxyl, phenols and naphthols such as paminophenol, p-nitrosophenol, 2-tert-butylphenol, 4-tert-butylphenol, 2,4-di-tert-butylphenol, 2-methyl-4-tert-butylphenol, 2,6-tert-butyl-4-methylphenol or 4-tert-butyl-2,6-dimethylphenol, quinones, for example hydroquinone or hydroquinone monomethyl ether, aromatic amines, for example N,N-diphenylamine, phenylenediamines, for example N,N′-dialkyl-p-phenylenediamine, where the alkyl radicals may be the same or different and each independently consist of 1 to 4 carbon atoms and may be straight-chain or branched, for example N,N′-dimethyl-p-phenylenediamine or N,N′-diethyl-p-phenylenediamine, hydroxylamines, for example N,N-diethylhydroxylamine, imines, for example methyl ethyl imine or methylene violet, sulfonamides, for example Nmethyl-4-toluenesulfonamide or N-tert-butyl-4-toluenesulfonamide, oximes such as aldoximes, ketoximes or amidoximes, for example diethyl ketoxime, methyl ethyl ketoxime or salicylaldoxime, phosphorus compounds, for example triphenylphosphine, triphenyl phosphite or triethyl phosphite, sulfur compounds, for example diphenyl sulfide or phenothiazine, metal salts, for example cerium (III) acetate or cerium (III) ethylhexanoate, or mixtures thereof.

Polymerization is preferably inhibited with phenothiazine, hydroquinone, hydroquinone monomethyl ether, 4-hydroxy-2,2,6,6-tetramethylpiperidine N-oxyl, 4-oxo-2,2,6,6-tetramethylpiperidine N-oxyl, 2,6-tert-butyl-4-methylphenol or mixtures thereof.

Very particular preference is given to using phenothiazine as polymerization inhibitor.

### EXAMPLES

### Example 1

Ethyl acrylate (99.88% by weight of ethyl acrylate, 0.05% by weight of isobutyl acrylate, 0.03% by weight of N,N′-di-sec-butyl-para-phenylenediamine, 0.01% by weight of 4-hydroxy-2,2,6,6-tetramethylpiperidinyloxyl) was metered in continuously beneath the first tray of a rectification column (diameter 1000 mm, 15 dual-flow trays) with a shell and tube heat exchanger (62 m2) in the bottom region and an external cooler in the top region. The rectification column was operated at a pressure of 400 mbar. The reflux ratio was 0.2. The reflux was stabilized with 4-hydroxy-2,2,6,6-tetramethylpiperidinyloxyl. The feed to the rectification column was 6094 kg/h. At the top of the rectification column, 7386 kg/h of distillate was removed.

The parts of the evaporator that were in contact with the product were made from stainless steel (1.4571 material according to DIN EN 10088:16.5% to 18.5% by weight of chromium, 10.5% to 13.5% by weight of nickel, 2.0% to 2.5% by weight of molybdenum, up to 0.7% by weight of titanium). The corrosion rate was less than 0.01 mm/a.

In the evaporator, no polymer deposits were apparent after 100 days.

### Example 2 (Comparative Example)

Ethyl acrylate (99.88% by weight of ethyl acrylate, 0.05% by weight of isobutyl acrylate, 0.03% by weight of N,N′-di-sec-butyl-para-phenylenediamine, 0.01% by weight of 4-hydroxy-2,2,6,6-tetramethylpiperidinyloxyl) was metered in continuously to the 39th tray of a rectification column (diameter 1100 mm, 52 dual-flow trays) with a shell and tube heat exchanger (51 m2) in the bottom region and an external cooler in the top region. The rectification column was operated at a pressure of 1000 mbar. The reflux ratio was 0.93. The reflux was stabilized with 4-hydroxy2,2,6,6-tetramethylpiperidinyloxyl. The feed to the rectification column was 6399 kg/h. 6094 kg/h of product was discharged from the bottom of the rectification column.

The parts of the evaporator that were in contact with the product were made from nonalloyed steel (1.0425 material: up to 0.3% by weight of chromium, up to 0.3% by weight of nickel, up to 0.08% by weight of molybdenum, up to 0.03% by weight of titanium). The corrosion rate was less than 0.01 mm/a.

In the evaporator, distinct polymer deposits were apparent after 100 days.

1.-12. (canceled)

13. A process for continuously distilling acrylates by means of a rectification column, wherein the acrylate content in the feed to the rectification column is at least 80% by weight, the liquid in the bottom region of the rectification column is heated by means of an evaporator, and the parts of the evaporator that are in contact with product are made from stainless steel.

14. The process according to claim 13, wherein methyl acrylate, ethyl acrylate, n-butyl acrylate or 2-ethylhexyl acrylate is used as acrylate.

15. The process according to claim 13, wherein the acrylate content in the feed to the rectification column is at least 85% by weight.

16. The process according to claim 13, wherein the acrylate content in the feed to the rectification column is at least 90% by weight.

17. The process according to claim 13, wherein the acrylate content in the feed to the rectification column is at least 95% by weight.

18. The process according to claim 13, wherein the evaporator is a shell and tube heat exchanger.

19. The process according to claim 13, wherein the parts of the evaporator that are in contact with product are made from stainless steel having 10.5% to 30.0% by weight of chromium.

20. The process according to claim 19, wherein the parts of the condensation column that are in contact with product are made from stainless steel additionally having 2.0% to 35.0% by weight of nickel.

21. The process according to claim 19, wherein the parts of the condensation column that are in contact with product are made from stainless steel additionally having 0.1% to 8.0% by weight of molybdenum.

22. The process according to claim 19, wherein the feed to the rectification column has an acid number of less than 100 mg of potassium hydroxide per g of feed.

23. The process according to claim 13, wherein the feed to the rectification column has an acid number of less than 10 mg of potassium hydroxide per g of feed.

24. The process according to claim 13, wherein the feed to the rectification column has an acid number of less than 1 mg of potassium hydroxide per g of feed.
